(19) United States
(12) Patent Application Publication
(10) Pub. No.: US 2021/0106450 A1
(43) Pub. Date: Apr. 15, 2021

(54) Joint Assistant:Outer Support for Inner Problems

(71) Applicant: Mary Elizabeth Colburn, Redondo Beach, CA (US)

(72) Inventor: Mary Elizabeth Colburn, Redondo Beach, CA (US)

(21) Appl. No.: 16/653,238

(22) Filed: Oct. 15, 2019

(51) Int. Cl. A61F 5/01

(52) U.S. Cl. CPC A61F 5/0111

(57) ABSTRACT

This invention is an ankle sprain compression support wrap to be used for physical activities. This is a bidirectional, adjustable ankle compression support wrap that may apply integrated compression, torque, leverage and tension vertically and horizontally, proximally and distally to the ankle joint, which connects perpendicularly or diagonally, wraps underneath the arch, around the shin and may be worn on the left or right ankle, and applied on the medial or lateral side. It is applied with the foot dorsiflexed four fingers above the malleolus with the shin wrap being held in place after it is wrapped around the leg once and the arch wrap secured to the opposing side of the shin wrap when aligned with the malleolus. The shin wrap further secures the arch wrap when it continues to wrap around the shin, which is secured by self attaching material. Tension may be adjusted according to the needs of the user using the self attaching material.

### BACKGROUND OF THE INVENTION

### 1. Field of the Invention

The present invention is in the field of ankle joint support by using tension, leverage and compression to support the joint. More specifically, it provides fully adjustable support of the ankle joint during physical activity while allowing full mobility by applying proximal and distal tension, compression, leverage, and torque from the perpendicularly or diagonally joined shin and arch wraps by wrapping around the shin and under the arch and attaching with self attaching material or sewn junctions. Since it is a bilateral device that can be used on the right or left ankle, the direction of the pull of the arch wrap and shin wrap determines the areas of support and leverage, which may be applied and adjusted as needed using the self attaching material. Furthermore, the concept of applying tension, leverage, compression and torque to stabilize the joints may be applied to all joints of the body using alternative embodiments of the present invention.

### 2. Description of Related Art

Ankle sprains commonly result in instability, weakness, and reinjury of the ankle joint due to surrounding tissue damage from inversion, eversion, and tears of sesmoidotic tissue. Severe ankle sprains, such as most high ankle sprains, and grade 3 sprains can be debilitating and may result in permanent damage and dysfunction of the joint. According the American Family Physician, an estimated 2 million ankle injuries occur per year in the US and many people do not seek a medical opinion when they occur (Douglas, 2006).

Many ankle braces or wraps either lack the support in places where it is needed or have too much support making it bulky and it often limits mobility. Many wraps or ankle braces may cause skin irritation, cuts or blisters. Bulkiness can make it difficult to wear with shoes, it can cause stretching of the shoes, and may not perform all of the said proper functions such as increasing stability of the ankle joint due to the bulkiness of the brace. These devices may cause injuries in other areas of the body due to limited mobility. Additionally, wraps that are more adjustable commonly have material that irritates the skin at the Achilles tendon or under the arch, especially when worn for long periods of time due to the constant movement of the ankle joint and being covered by the ankle wrap or brace. Since everyone's biomechanics, body shapes and sizes are different, there is a need for a completely adjustable support device that may allow for more support in the areas of each particular person's need. Therefore, what is clearly needed is an ankle wrap and method of applying it that solves the problems mentioned above. Ivins, Douglas M. D., M.S.C.E, University of Oklahoma College of Medicine-Tulsa, Tulsa, Okla. American Family Physician. 2006 Nov. 15; 74 (10):1714-1720

### SUMMARY OF THE INVENTION

The bidirectional, adjustable ankle compression support wrap for enhancing ankle joint support is comprised of a thin, elastic material that wraps horizontally around the upper portion of the shin. It is further comprised of an arch wrap that is attached to the shin wrap that wraps underneath the arch and attaches to the horizontal shin wrap which is further secured by continued covering of the shin wrap. The method of application of the wraps may apply fully adjustable integrated compression, leverage, torque and tension vertically and horizontally, proximally and distally to the ankle joint, which connects perpendicularly or diagonally and wraps under the arch and around the shin and may be worn on the left or right ankle on the medial or lateral side.

The optimal fit of the ankle compression support wrap requires adjustments to be made by the user in order to, attain optimal movement and functionality of both the wrap and the ankle joint. The ankle compression support wrap is effective for encircling the girth of the lower leg distally to the medial and lateral malleolus and under the arch of the foot when aligned with the malleolus with the foot dorsiflexed then fastened via self attaching material. The said device has the potential to assist the calcaneus and talus to track and function properly after a severe high ankle sprain during physical activity. Furthermore, it may allow multidirectional mobility during physical activity in the frontal, transverse, and sagittal planes.

The method of application of the ankle compression support wrap, according to claim 3, has the potential to be worn for long periods of time during physical activity on many different types of terrain barefoot or with shoes, sandals, or other footwear on areas such as sand or grass, or floors without causing blisters or skin irritation if the wraps are adjusted properly. The device is recommended to be placed on the foot barefoot in the dorsiflexed position for best results and may be worn with socks and/or shoes over it. The attachments of the shin and arch wraps may provide support by absorbing forces medially, laterally, frontally, diagonally and distally through the tension, compression and junctions applied by the wraps. When the shin wrap is applied distally from the medial malleolus and wrapped clockwise with the arch wrap applied medially and attaching laterally with the foot dorsiflexed, the areas of potential support may include the anterior tibiofibular ligament, the anterior talofibular ligament, the calcaneofibular ligament, the tibiocalcaneal ligaments, and the peroneal tendons. The amount of possible support and compression depend on the adjustments of the shin and arch wraps. When the wrap is applied laterally, the potential support provided to the medial ligaments may include the deltoid complex, the plantar calcaneonavicular ligament, the talonavicular ligament, and the medial talocalcaneal ligament.

The shin and arch wraps could be connected diagonally or perpendicularly and could be sewn or connected via self attaching material on one or both sides to allow for greater adjustability. The lengths and widths of the wraps may vary depending on the area of the body for which stabilization is needed. The self attaching material may appear on any end or part of the elastic for which it is needed to better secure the wrap. The wrap may include self attaching material on outside of the beginning end of the shin wrap, or the shorter end to allow for easier adjustability of the shin wrap and connection of the arch wrap. The concept of using the proximal and distal areas of the joint with specific areas of compression without bulky materials may allow for a thin adjustable hard plastic or fabric insertion on the shin wrap to aide with maintaining the position of the shin and hard supportive material on the arch wrap for further support. The concepts of using proximal and distal tension with the wraps and self attaching material may be applied to other joints in the body using various widths and lengths of the material with different attachment points. The width of the material may vary depending on the joint it is designed to support. The width of the wraps may consist of one half to 3 inch wide wraps with self attaching material on any end or part needed to support the area of the body for which it is designed. The length of the material may be as long as 30 inches and as short as 6 inches.

### BRIEF DESCRIPTION OF THE DRAWINGS

FIG. 1 is a flat laid out frontal view of the ankle sprain compression support wrap according to an embodiment of the present invention.

FIG. 2 is a flat, laid out dorsal view of the ankle sprain compression support wrap according to an embodiment of the present invention.

FIG. 3 is a medial view of the ankle sprain compression support wrap completely wrapped according to an embodiment of the present invention.

FIG. 4 is a lateral view of the ankle sprain compression support wrap completely wrapped according to an embodiment of the present invention.

FIG. 5 is a dorsal view of the ankle sprain compression support wrap completely wrapped according to an embodiment of the present invention.

FIG. 6 is a frontal view of the ankle sprain compression support wrap completely wrapped according to an embodiment of the present invention.

FIG. 7 is a medial application view of the ankle sprain compression support wrap according to an embodiment of the present invention.

FIG. 8 is a lateral application view of the ankle sprain compression support wrap according to an embodiment of the present invention.

FIG. 9 is a flat laid out frontal view of the ankle sprain compression support wrap according to a modified embodiment of the present invention.

FIG. 10 is a flow diagram of the order of optimal application of the wrap.

### DETAILED DESCRIPTION OF THE INVENTION

The inventor provides a unique wrap that uses adjustable leverage, tension, and torque to provide support for ankle sprains that is placed well above the ankle joint as opposed to on or near the ankle joint. The present invention is described in detail in the following examples, which may present one embodiment of the invention.

FIG. 1 is a flat laid out frontal view of the ankle sprain compression support wrap according to an embodiment of the present invention. In FIG. 1, the wrap consists of a thin, flexible, elastic material that is approximately three inches wide with self attaching material on the ends 3, 4 that attach to the wrap. A sewn perpendicular or diagonal attachment (not shown) 5 with self attaching material that attaches at the proximal, short end or beginning of the shin wrap, goes under the arch and attaches on the opposite side of the shin wrap four fingers above the malleolus using the self attaching material on the arch wrap 2. The beginning of the shin wrap 1 has a nonirritating fabric covering 6. The perpendicular or diagonal attachment point (not shown) 5 is sewn or uses a self attaching material approximately 2 inches from the beginning of the shin wrap. The opposite end of the shin wrap 3 has a self attaching material. Once the shin wrap 1 has been wrapped once around the shin and held in place four fingers above the malleolus, the arch wrap 2 goes underneath the arch of the foot with the foot dorsiflexed and fastens with self attaching fabric 4 to the opposing side of the shin wrap aligned with the medial malleolus. The self attaching material 3, 4 attaches via hooks of self attaching material. The shin wrap 1 of the wrap spans approximately 18 to 30 inches and is approximately 3 inches wide. The differences in size account for different sizes of people. The arch wrap 2 portion of the wrap spans approximately 8 to 20 inches and is approximately 3 inches wide. The perpendicular 5 or diagonal junction 91 of the wrap of the shin and arch wrap 2 is between two and four inches from the beginning or short end of the shin wrap, 1 and sewn 5 or attached via self attaching material approximately one inch from the top of the shin wrap 1.

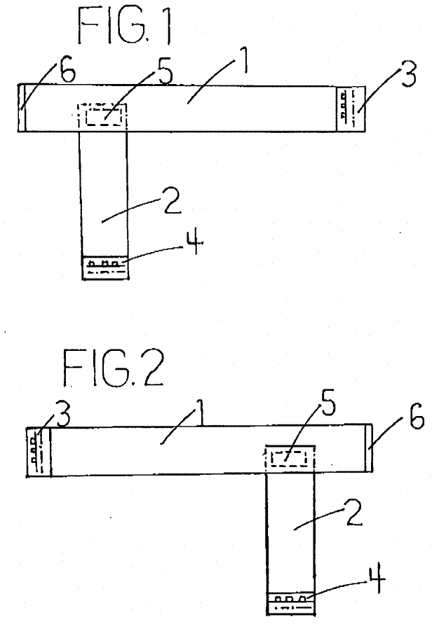

FIG. 2 is a flat, laid out dorsal view of the ankle sprain compression support wrap according to an embodiment of the present invention. This view illustrates how it can be used for the opposing limb or side. The perpendicular attachment 5 is sewn or attached via self attaching material (not shown) so it is nonirritating and may be used on either side.

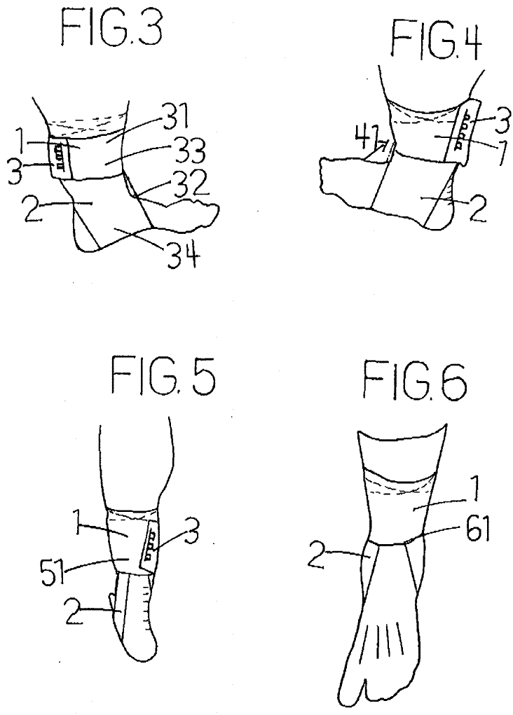

FIG. 3 is a medial view of the ankle sprain compression support wrap completely wrapped medially around the left foot according to an embodiment of the present invention. The shin wrap 1 has covered the arch wrap 2 and is securely fastened with the self attaching material 4. In doing so, there is now vertical tension 32 coming from the arch wrap, 2 horizontal compression 31 coming from the shin wrap, 1 and torque 33 from the junction of the shin and arch wraps intersecting. There is slight sagittal tension 34 on the medial side from the arch wrap 2.

FIG. 4 is a lateral view of the ankle sprain compression support wrap completely wrapped according to an embodiment of the present invention. The shin wrap 1 has covered and further secured the arch wrap 2 and is securely fastened with the self attaching material 3 and covers the arch wrap 2. There is adjustable leverage and vertical tension of the dorsiflexed foot 41 of the medial and lateral aspect of the arch wrap 2.

FIG. 5 is a dorsal view of the ankle sprain compression support wrap completely wrapped according to an embodiment of the present invention. The shin wrap 1 is completely fastened via the self attaching material 3. The positioning of the shin wrap 1 is well above the Achilles tendon, allowing for greater mobility and less risk of skin irritation if adjusted properly. The horizontal compression and secured arch wrap allows for complete adjustability 51.

FIG. 6 is a frontal view of the ankle sprain compression support wrap completely wrapped according to an embodiment of the present invention. The arch wrap 2 is applied four fingers above the malleolus and aligned with the medial and lateral malleolus. It is then secured to the shin wrap, 1 which is above the ankle joint in the frontal view, allowing for a lower risk of skin irritation frontally and support using the leverage created by the adjustable intersections and tension of the wraps. There is torque, vertical and horizontal tension, and compression created at a junction 61 of the wrap. Additionally, due to the high junction point, skin irritation is much less likely due to the lack of movement in that area of the junction.

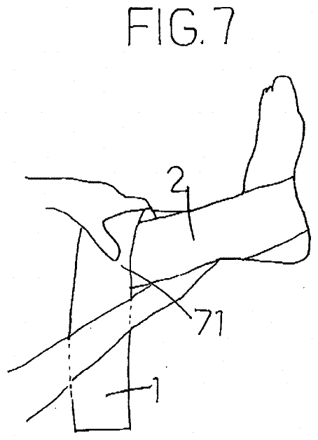

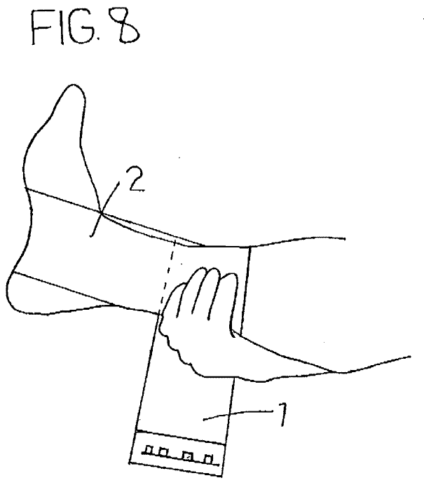

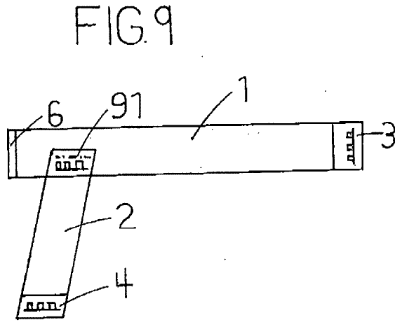

FIG. 7 is a medial view of the application of the ankle sprain compression support wrap according to an embodiment of the present invention. One hand holds the shin wrap 1 in place while the other attaches the arch wrap 2. An application well above the areas of movement of the ankle 71 allows for full mobility and less likelihood of skin irritation. The shin wrap 1 is applied approximately four fingers above the medial or lateral malleolus. The wrap may be applied medially or laterally, on the right or left ankle and may be attached perpendicularly or diagonally.

FIG. 8 is a lateral view of the application of the ankle sprain compression support wrap according to an embodiment of the present invention.

FIG. 9 is a flat laid out frontal view of the ankle sprain compression support wrap according to a modified embodiment of the present invention. It uses self attaching material for the arch wrap on both sides and is attached diagonally instead of vertically.

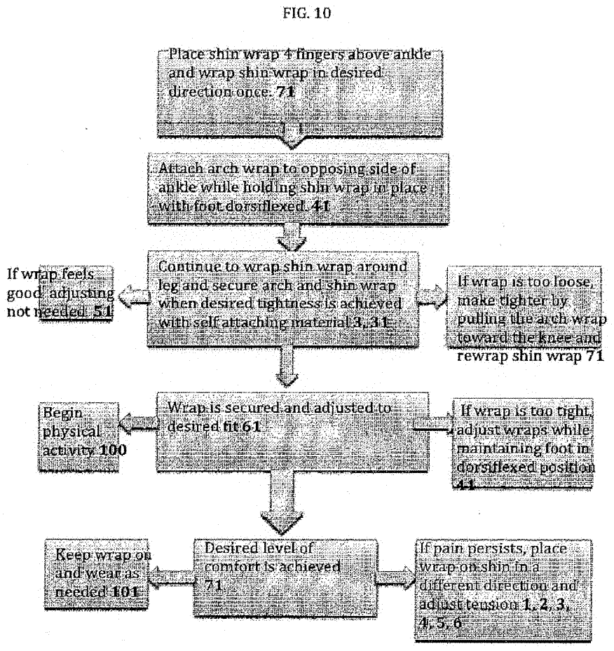

FIG. 10 shows a flow diagram of the order of optimal application of the wrap. It explains possible problems and solutions to application of the wraps and optimal usage of the wrap. Box 71 describes the optimal application location of the shin wrap. Place shin wrap 4 fingers above ankle and wrap shin wrap in desired direction once with desired tightness 31.

Box 41 describes optimal placement of the arch wrap. Attach arch wrap to opposing side of ankle while holding shin wrap in place with foot dorsiflexed 41. Box 3, 31 describes optimal application of the arch wrap. Continue to wrap shin wrap around leg and secure arch and shin wrap when desired tightness is achieved with self attaching material. Box 51 describes a common situation with the wraps. Box 71 describes a solution to a common issue with the arch and shin wrap. If wrap is too loose, make tighter by pulling the arch wrap toward the knee and rewrap shin wrap. Box 61 describes a proper adjustment and fit of the wraps. Wrap is secured and adjusted to desired fit. Box 100 describes the ability to perform physical activity after optimal fitting. Begin physical activity. Box 41 describes a common situation encountered with the wraps. If wrap is too tight, adjust wraps while maintaining foot in dorsiflexed position. Box 71 describes desired level of comfort. Desired level of comfort is achieved. Box 101 describes wearability. Keep wrap on and wear as needed. Box 1, 2, 3, 4, 5, 6 describes a possible solution to a possible encounter with the wraps. If pain persists, place wrap on shin in a different direction and adjust tension.

It will be apparent to one with skill in the art of ankle wraps and braces that the usage of the inventions may be provided using some or all of the mentioned features and components without departing from the spirit and scope of the present invention. It will also be apparent to the skilled artisan that the embodiments described above are specific examples of a broader invention, which may have greater scope than any of the singular descriptions explained. There may be many alterations in the descriptions without departing from the spirit and scope of the present invention.

I claim:

1. A bidirectional, adjustable ankle compression support wrap for enhancing ankle joint support comprising:
A thin, elastic material that wraps horizontally around the shin.
An arch wrap that is attached to the shin wrap that wraps underneath the arch and attaches to the horizontal shin wrap, which is further secured by continued covering of the shin wrap.
The method of application of the wraps may apply fully adjustable integrated compression, leverage, torque and tension vertically and horizontally, proximally and distally to the ankle joint, which connects perpendicularly or diagonally and wraps under the arch and around the shin and may be worn on the left or right ankle on the medial or lateral side.

2. The ankle compression support wrap, according to claim 2, wherein the optimal fit of the wrap and adjustments which are required to be made by the user in order to attain optimal movement and functionality of both the wrap and the ankle joint.

3. The ankle compression support wrap, according to claim 3, is effective for encircling the girth of the lower leg distally to the medial and lateral malleolus with the foot dorsiflexed and under the arch of the foot when aligned with the malleolus then fastened via self attaching material.

4. The ankle compression support wrap, according to claim 3, has the potential to assist the calcaneus and talus to track and function properly after a severe high ankle sprain during physical activity.

5. The ankle compression support wrap, according to claim 2, has the potential provide additional support during moderate to vigorous levels of physical activity and may allow multidirectional mobility during physical activity in the frontal, transverse, and sagittal planes.

6. The method of application of the ankle compression support wrap, according to claim 3, has the potential to be worn for long periods of time during physical activity on many different types of terrain barefoot or with shoes, sandals, or other footwear on areas such as sand or grass, or floors or other surfaces without causing blisters or skin irritation if the wraps are adjusted properly.

7. The ankle compression support wrap, according to claim 3, in said application of the device: The arch wrap is recommended to be placed on the foot barefoot in the dorsiflexed position for best results and may be worn with socks and/or shoes over it.

8. The ankle compression support wrap, according to claim 1, herein said: The attachments of the shin and arch wraps may provide support by absorbing forces medially, laterally, frontally, diagonally and distally through the tension, compression and junctions applied by the wraps.

9. The ankle compression support wrap, according to claim 1, herein said: When the shin wrap is applied distally from the medial malleolus and wrapped clockwise with the arch wrap applied medially and attaching laterally with the foot dorsiflexed, the areas of potential support may include the anterior tibiofibular ligament, the anterior talofibular ligament, the calcaneofibular ligament, the tibiocalcaneal ligaments, and the peroneal tendons. The amount of possible support and compression depend on the adjustments of the shin and arch wraps.

10. The ankle compression support wrap, according to claim 1, herein said: When the wrap is applied laterally, the potential support provided to the medial ligaments may include the deltoid complex, the plantar calcaneonavicular ligament, the talonavicular ligament, and the medial talocalcaneal ligament.

11. The ankle compression support wrap, according to claim 1, herein said: The shin and arch wraps could be connected diagonally or perpendicularly and could be sewn or connected via self attaching material on one or both sides to allow for greater adjustability. The lengths and widths of the wraps may vary depending on the area of the body for which stabilization is needed. The self attaching material may appear on any end or part of the elastic for which it is needed to better secure the wrap.

12. The ankle compression support wrap, according to claim 1, herein said: The wrap may include self attaching material on outside of the beginning end of the shin wrap, or the shorter end to allow for easier adjustability of the shin wrap and connection of the arch wrap.

13. The concept of using the proximal and distal areas of the joint with specific areas of compression as said in claim 1 without bulky materials may allow for a thin adjustable hard plastic or fabric insertion on the shin wrap to aide with maintaining the position of the shin and hard supportive material on the arch wrap for further support.

14. The concepts of using proximal and distal tension with the wraps and self attaching material may be applied to other joints in the body using various widths and lengths of the material with different attachment points. The width of the material may vary depending on the joint it is designed to support. The width of the wraps may consist of one half to 3 inch wide wrap with self attaching material on any end or part needed to support the area of the body for which it is designed. The length of the material may be as long as 30 inches and as short as 6 inches.
